(19) United States
(12) Patent Application Publication
(10) Pub. No.: US 2016/0197212 A1
(43) Pub. Date: Jul. 7, 2016

(54) Processing of Nanoparticles for Improved Photocells and Drug Delivery

(71) Applicant: WISYS Technology Foundation, Inc., Madison, WI (US)

(72) Inventor: Jennifer A. Dahl, Eau Claire, WI (US)

(21) Appl. No.: 14/987,039

(22) Filed: Jan. 4, 2016

Related U.S. Application Data
(60) Provisional application No. 62/099,758, filed on Jan. 5, 2015.

(51) Int. Cl. H01L 31/0224; A61N 5/02; H01L 31/068

(52) U.S. Cl. CPC H01L 31/022491; H01L 31/068; A61N 5/02; A61N 5/025

(57) ABSTRACT

The manipulation of nanoparticles is facilitated through the use of a Langmuir-Blodgett trough constraining the nanoparticles to two dimensions and allowing their density to be controlled through the barriers of the Langmuir-Blodgett trough. A film formed in this manner can be applied to enhance a transparent conductive electrode on the photovoltaic cell. Alternatively the nanoparticles as so constrained can be given two types of functionalization, for example, of an anticancer agent and a targeting ligand.

### CROSS REFERENCE TO RELATED APPLICATION

This application claims the benefit of U.S. provisional application 62/099,758 filed Jan. 5, 2015, and hereby incorporated, in its entirety by reference.

### BACKGROUND OF THE INVENTION

The present invention relates to a method of processing nanoparticles for use in multiple applications.

Recent decades have brought about reliable, methods for the synthesis and functionalization of metal nanoparticles but applying these nanoparticles to many important applications has been hindered by the difficulty of organizing the particles into regular structures with controlled interparticle spacing, for example, as needed to optimize electronic conductivity. Similarly, precise interparticle spacing can be important to control the optical transport properties of these nanoparticles.

It is generally known to functionalize nanoparticles for the purpose of applying a targeting ligand to the nanoparticles encouraging accumulation of the nanoparticles, for example, in a tumor. The nanoparticles may then serve, for example, as selective absorbers of radiation (for example, microwave radiation) for hyperthermic treatments.

### SUMMARY OF THE INVENTION

The present invention provides: (1) a method of constructing films of nanoparticles with controlled interparticle spacing for incorporation into structures such as solar cells and (2) a method of restraining nanoparticles for sophisticated functionalization, for example, using two different functionalizing agents of an anticancer agent and a targeting agent on each nanoparticle so that nanoparticles can be delivered to a tumor site with an anticancer agent that may be activated by heat from the nanoparticles.

These particular objects and advantages may apply to only some embodiments falling within the claims and thus do not define the scope of the invention.

### BRIEF DESCRIPTION OF THE DRAWINGS

FIG. 1 is a flowchart and associated diagram showing the fabrication of a transparent conductive oxide solar cell augmented with gold nanoparticles;

FIGS. 2a-c are representations of functionalized gold nanoparticles on an air water interface (shown in cross-sectional elevation), the nanoparticles functionalized for cross-linking to limit diffusion and rotation;

FIG. 3 is a figure similar to that of FIG. 2 showing introduction of a water-soluble functional group to only one side of the immobilized nanoparticles;

FIG. 4 shows bi-functionalized nanoparticles such as may provide for both an anticancer agent and a targeting agent.

### DETAILED DESCRIPTION OF THE PREFERRED EMBODIMENT

### Photovoltaic Cell

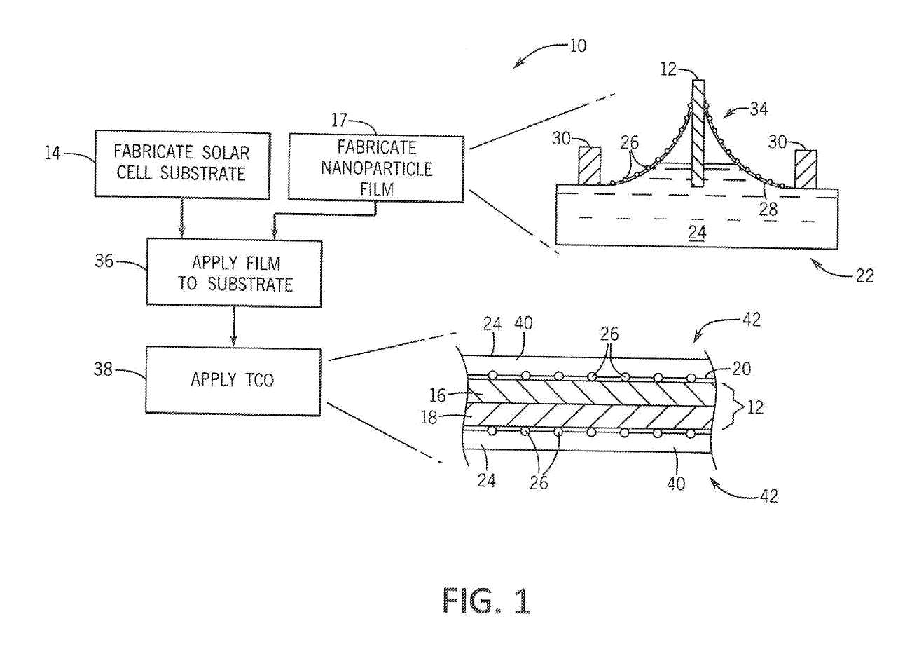

Referring now to FIG. 1, a process 10 for fabrication of photocells ma provide for the construction of a solar cell substrate 12 per process block 14, for example, including assembling in parallel adjacent layers of dissimilarly doped semiconductor elements 16 and 18 forming a PN junction.

In parallel with the above process and possibly in a different facility that does not need traditional integrated circuit processing technologies and which would not suffer from contamination problems caused by nano conductors, a nanoparticle film 20 may be fabricated as indicated by process block 17. This film 20 may be fabricated, for example, through the use of a Langmuir trough 22 of the type having a tray holding water 24 on whose surface functionalized hydrophobic nanoparticles 26 may be introduced, for example, in a solution of hexanes to arrange themselves along an air water interface 28.

Nanoparticles 26, fur example, may be gold nanoparticles functionalized with thiols, for example, providing the hydrophobic property. The nanoparticles 26 may have a size varying from 3 to 8 nanometers and may be synthesized using a Brust synthesis. Purification to eliminate excess thiols and phase transfer reagents is performed using the Soxhlet extraction technique.

Barriers 30 on the trough 22 corralling the nanoparticles 26 along the surface of the water 24 may then be isometrically contracted together to compress the nanoparticles 26 into a desired film having a controlled average interparticle spacing and cross-linking agents such as a dithiol or bisdithiol or mixture of the same (and alternatively possibly being diphosphines or diamines) may then be introduced to form cross-links between the nanoparticles 26 connecting them into a robust monolayer 34. An upper or lower surface of the monolayer 34 may then be modified to make it hydrophilic as will be discussed below, with the cross-linking preventing diffusion of the nanoparticles 26 into the water 24.

The solar cell substrate 12, for example, having an outer surface of silicon oxide or silicon nitride, can be drawn through the monolayer 34 which then adheres to the broad faces of the substrate 32, utilizing the Langmuir-Blodgett method of monolayer transfer to a solid substrate. For example when the lower surface of the monolayer 34 is made hydrophilic, when the substrate 32 is drawn vertically out of the water 24 through the air water interface 28 a monolayer 34 will be deposited and adhered on its opposite surfaces. During this withdrawal, the barriers 30 may be moved together to preserve the desired density of nanoparticles 26. Conversely, the upper surface of the monolayer 34 may be made hydrophilic and the substrate passed vertically downward through the air water interface 28. The result is a thin film with a controlled spacing of nanoparticles 26 adhered to the upper surface of the substrate 12 opposite the backer electrode 21. The same result may also be achieved using a Langmuir-Schaffer transfer, by which a substrate is oriented parallel to the air-water interface, lowered until contact is made, and then withdrawn from the interface, leaving the hydrohphilic side of the film bonded to the substrate.

At process block 38, a layer of a transparent conductive oxide 40 (for example, indium tin oxide) may be sputtered over the nanoparticles 26 to provide a composite conductive electrode 42 on opposite sides of the solar cell substrate 12 for collection of electrical current and the driving of a load. The gold nanoparticles 26 provide improved capture of light energy either by absorption and retransmission or internal reflection. A gram of gold nanoparticles can provide coating for 4000 square meters of transparent conductive oxide. Alternatively, the crosslinked film of gold nanoparticles may be applied to a previously constructed solar cell, where the film would prevent captured light from escaping the cell, thus increasing the dwell time of the incoming radiation and in turn increasing photocarrier generation.

### Drug Delivery System

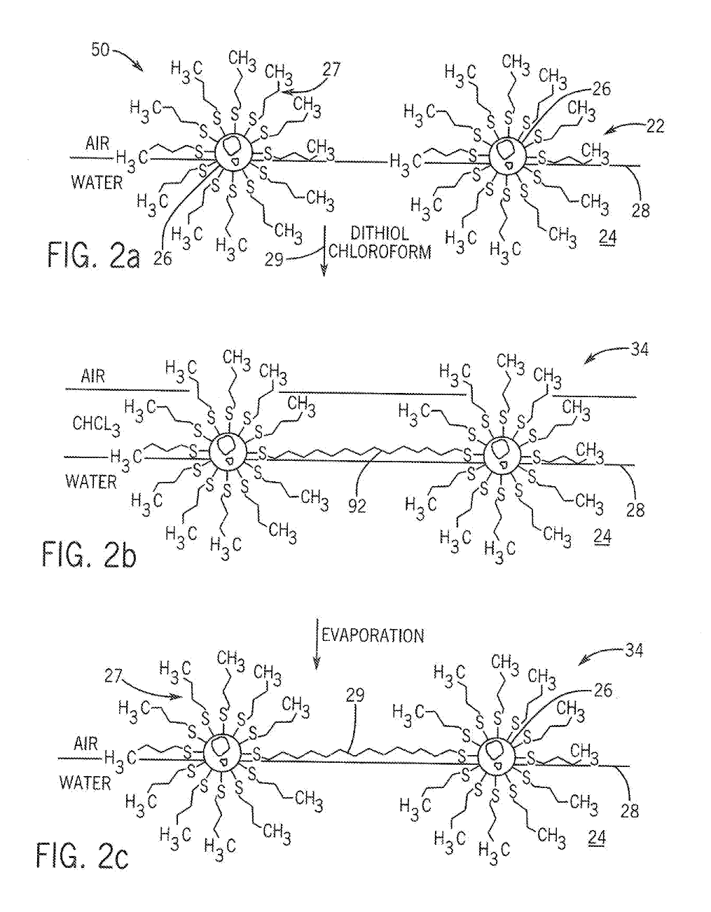

Referring now to FIGS. 2a-c, thiol functionalized gold nanoparticles 26 in the Langmuir-Blodgett trough 22 described above may be compressed together to get the desired separation as indicated by FIG. 2. The thiol functionalization 27. as discussed above, provides a hydrophobic quality to the gold nanoparticles 26 causing them to align along the air water interface. The nanoparticles 26 may be compressed to desired density using the barriers 30 (shown in FIG. 1) and joined into a monolayer 34 with a cross-linking agent 29 being for example a dithiol or bisdithiol or combination for example as described in paper [1] cited below and hereby incorporated by reference. The cross-linking agent 29 may be introduced in a layer of chloroform (CHCl₃) may be applied over the water 24 (as shown in. FIG. 2b) and then allowed to evaporate as shown in FIG. 2c to promote a cross-linking of the spaced nanoparticles 26. A polar peptide on the cross-linking agent 29 helps the agent lie flat on the surface of the water 24. This cross-linking provides two benefits of restricting rotation of the nanoparticles 26 and preventing their diffusion into the water 24 as would otherwise occur when there hydrophobic nature is modified by a ligand exchange process discussed below.

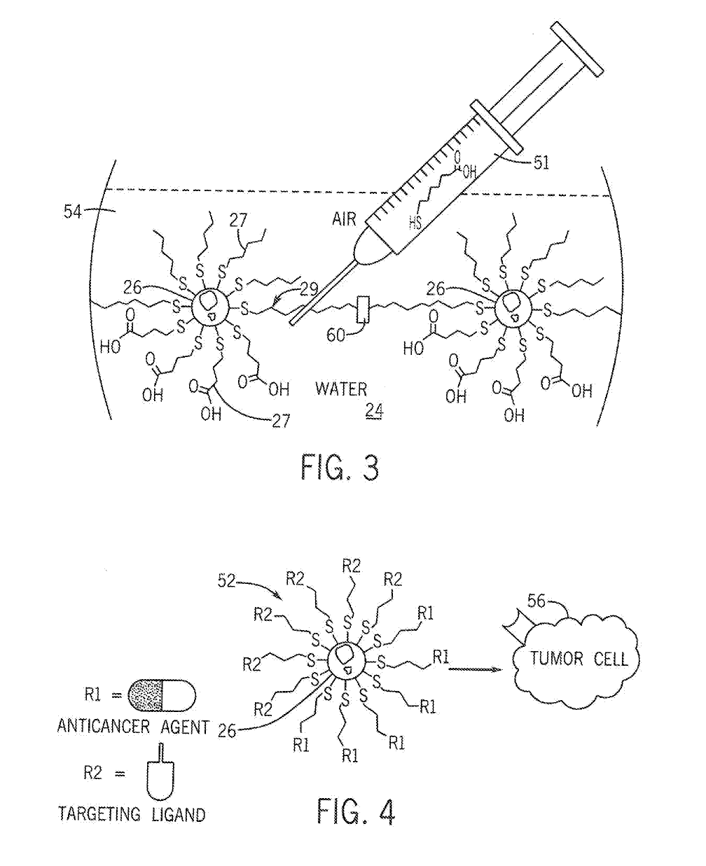

Referring now to FIG. 3, after this cross-linking, new functionalization groups 51 can be introduced into the water 24 in contact with the previous functionalization of the nanoparticles 26 with the thiol functionalization to provide an exchange of ligands. For example, an ethanolic solution of mercaptohexanoic acid can be injected into the aqueous sub phase (the water 24) and the excess mercaptohexanoic acid will displace the short chain alkanethiol via a ligand exchange process to provide for asymmetric functionalization of each nanoparticle 26. This particular ligand exchange provides a hydrophilic surface to the monolayer 34.

Conversely, the distal ends of the upper thiol functionalization of each nanoparticle 26 may be modified by through the introduction of a carrier fluid 54 (for example hexanes) over the surface of the water 24 providing a layer immiscible with the water 34 but acting as a solvent to hold the ligands for exchange with the upper thiol functionalization. Generally, the carrier fluid 54 is selected to be non-soluble in the water 24 but to provide a solvent for the desired ligand for exchange.

Referring now to FIG. 4, this regioselective ligand exchange process may be used to produce a so-called Janus nanoparticle 52 having different sides with different functionalizations R1 and R2. In one case, one functionalization may provide for a hydrophilic side to the monofilm 34 for attachment to the substrate 12 discussed above.

Alternatively, one functionalization (R1) may provide for an anticancer agent that works in conjunction with hyperthermia treatment made possible using the nanoparticle 26 and the other functionalization (R2) may be a targeting ligand allowing the Janus nanoparticles 52 to be preferentially retained in a tumor 56 where the anticancer agent and heat therapy may be delivered.

Referring again to FIG. 3, for this latter purpose, a photo-cleavable cross-linking element 60 may be incorporated into the cross-linking between nanoparticles 26 to allow individual nanoparticles 26 to be extracted by light exposure. For example the photo-cleavable cross-linking element 60 may be nitrobenzyl photo-cleavable linker introduced in the hydrocarbon chain between the sulfur groups of the cross-linking agent 29.

These techniques can be extended to arrays of anisotropic nanomaterials such as nanorods, whose optical properties are expected to be dependent upon final orientation (either side-by-side or end--to-end) of the finished crosslinked composition. It will be generally understood that various techniques can be applied to the gold nanoparticles 26 to improve their ability to absorb radiation and in particular lower frequency microwave radiation as opposed to visible light radiation. These techniques increase the electrical size of the nanoparticles 26 for example through the use of a shell structure (nano shell).

The present application hereby incorporates the following materials in their entirety by reference:
[1] Langmuir isotherms of flexible, covalently crosslinked gold nanoparticle networks: Increased collapse pressures of membrane-like structures by Tayo A. Sanders II, Mariah N. Sauceda, Jennifer A. Dahl Materials Letters 04/2014; 120:159-162. DOI: 10.1016/j.matlet.2014.01.056; and
[2] Microwave synthesis of a bimodal mixture of triangular plate and spheroidal silver nanoparticles by Anneliese E. Laskowski Daniel A. Decato Mitchel S. Strandwitz Jennifer A. Dahl MRS Communications, 05/2015; 5(2):1-7. DOI: 10.1557/mrc.2015.23.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended, to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

What we claim is:

1. A method of fabricating a photovoltaic cell using the steps of:
(a) arranging nanoparticles in a monolayer with a controlled interparticle spacing on one surface of the photovoltaic cell; and
(b) applying a transparent conductive oxide over the monolayer to provide an electrode for the photovoltaic cell.

2. A method of drug delivery using the steps of
(a) applying a first and second functionalization to a plurality of nanoparticles;
(b) introducing the nanoparticles into a patient; and
(c) using an external radiation source to heat the nanoparticles as may accumulate in a tumor;
wherein the first functionalization is an anticancer agent for disrupting cancer cells and the second functionalization is a targeting agent for concentrating the nanoparticles in the tumor.
